(19) United States
(12) Patent Application Publication
(10) Pub. No.: US 2003/0060796 A1
(43) Pub. Date: Mar. 27, 2003

(54) Medical containers

(76) Inventors: Gunnar Andersson, Sollentuna (SE); Des Mulligan, Co Donegal (IE); Vittorio Sala, Ivera (IT)

Correspondence Address: PATENT ADMINSTRATOR, KATTEN MUCHIN ZAVIS ROSENMAN, 525 WEST MONROE STREET, SUITE 1600, CHICAGO, IL, 60661-3693, US

(21) Appl. No.: 10/132,271

(22) Filed: Apr. 26, 2002

Related U.S. Application Data
(62) Division of application No. 09/110,174, filed on Jul. 6, 1998, now abandoned.
(60) Provisional application No. 60/053,443, filed on Jul. 22, 1997.

(30) Foreign Application Priority Data
Jul. 8, 1997 (SE) 9702636-3

(51) Int. Cl. A61B 19/00

(52) U.S. Cl. 604/403

(57) ABSTRACT

The invention relates to a port system for establishing fluid communication with a container for storing medical fluids and a device for adding or withdrawing fluids from said container comprising at least one port having a sealed front end and a rear end open to the inside of said container and a base plate attachable to said container, wherein said port has a penetrable membrane serving as a barrier to the stored fluids. The port comprises a cap in its front end which axially extends into an essentially sleeve formed part provided with said penetrable membrane which is scaled from contamination by said cap and furthermore it is provided with means to expose said penetrable membrane when it is desired to establish fluid communication with the container.

### FIELD OF INVENTION

The present invention relates to a port system suitable as an opening for flexible medical containers which admits repeated entries into the container with a piercing device to establish fluid communication. The new port system has improved safety from contamination, is highly convenient and is manufactured with a simplified method.

### BACKGROUND OF THE INVENTION

The efforts of developing containers of polymeric materials for parenterally administerable medical products to replace glass bottles are described in the International patent application WO 95/08317 and in the as yet unpublished Swedish Patent Application SE 9601348-7. To find acceptance with authorities giving approvals to medical systems, such polymeric containers after being filled and finally sealed must be capable of being sterilized by high pressure steam (i.e. autoclavation) with a maintained barrier capacity against the environment and safety from migration of potentially hazardous agents. Moreover, the container must maintain a suitable aesthetic value and from an environmental protective view point be easy to recycle after use.

A particular problem in the development of this type of container is to provide it with a suitable opening or port that allows collection or administration of its stored fluids as well as supplementation of such complementary agents that are degradable during storage. The opening must admit repeated entries by devices for establishing fluid communication with the container, such as syringes, cannulas and spikes of infusion sets. Furthermore, the opening must be capable of withstanding several types of sterilization and provide a contamination free entrance of said devices for establishing fluid connection, so the fluids of the container not are wasted by microbial growth. The conventional bottle form or similar polymeric container comprises a pierceable rubber stopper sealingly fitted in an upper part of a neck-formed opening having a flange over which a removable protective foil of metal or polymers is sealed. In polymeric bags for infusion, a removable cap of rubber protects a membrane which can be penetrated by a spike to an infusion set. A drawback with these arrangements is that the penetrable upper (outer) surface of the stopper or the membrane might be contaminated before its sealing with a foil or a cap and although the finally sealed and filled container is autoclaved, this part will never be properly reached by the sterilizing steam. In order to overcome this problem and to provide more safe ports for flexible polymeric containers, means for temporarily introducing sterilizing steam to the mentioned parts are disclosed in the Swedish Patent Application No. 9601540-9. The mentioned conventional form of openings or ports to polymeric container also have the drawback that the rubber parts not are possible to recirculate together with the remaining plastic container, but has to be collected separately.

In order to improve on existing closure devices for containers, a new two color mold method is disclosed in the Swedish Patent Application No. 9700597-9 for preparing stoppers of an elastomeric material with enhanced resealing characteristics.

The container openings or ports disclosed in the mentioned documents suffer from complicated production procedures wherein the elastomer stopper is molded or inserted into a carrier which thereafter must be positioned into the mouthpiece of the port which is scaled by a cap or a foil, whereupon the port can be attached to the container or the flexible material for shaping a container. A reduction of production steps will both enable a cheaper process that also can provide more aseptic production conditions.

EP 0 097 054 (Hantakki Oy) discloses a flexible bag for medical fluids provided with an injection port which has a closure comprising a shielded resilient pierceable pad which reseals after penetration and which also may be protected from the stored fluids. This type of closure has the drawback in that it requires a complicated manufacturing and is difficult to keep aseptic in all its parts.

U.S. Pat. No. 4,303,067 (American Hospital Supply Corp.) describes an additive port for a medical bag through which medicals can be supplied by an injection device. The port has a pierceable, resealable plug made of an elastomer. Nothing is disclosed about the resealing capacity or the sterilizability of the closure.

U.S. Pat. Nos. 4,975,308 and 5,247,015 describe molded stoppers for blood tubes made of a halobutyl rubber dispersed in a mixture of polyolefins and a thermoplastic elastomer. No disclosures are given herein about their resealing capacity after needle penetration or their capacity of being heat sterilized.

There are numerous other citations in the literature of openings for pharmaceutical containers of various types, both made of glass or of polymeric materials which are disclosed as suitable to seal stored medical fluids. For fluids aimed for parenteral fluids (i.e. intravenous injection), there are requirements from medical authorities that the openings are capable of maintaining a barrier against the environment, both during sterilization by steam and during subsequent long term storage. It is also required that this type of closures shall be capable of resealing spontaneously and immediately after the withdrawal of a penetrating needle. They must also be able to reseal after multiple entries by penetrating needle, a cannula or a similar penetrating device to collect fluid from the container, or for adding a component to be mixed with the contents of the container. An improper resealing of the closure after penetration potentially will waste the integrity of stored fluids by providing a channel for microbial growth into the container. It is also a requirement that the container must not leak when its closure is penetrated during fluid connection, for instance during infusion to a patient through a cannula. Furthermore, the opening must be compatible with stored fluids and no migration of potentially hazardous agents from any of its parts is allowed. The opening must also withstand conventional sterilization processes including autoclavation at 121° C. or sterilization by means of irradiation without losing any of its sealing capacity. It is also a requirement by an increasing amount of medical authorities that each authorized container must be possible to recirculate and its therefore a demand that empty containers shall be possible to dispose without laborious disassembling and sorting of parts for individual recycling processes.

An object of the present invention is to provide a container port system which is manufactured with a technique that is sufficiently aseptic so a separate sterilization process is not needed before attaching to the container or the flexible material intended to form the container.

It is also an object of the present invention is to limit the lumber of process steps required to manufacture the port.

Another object of the invention is to provide a port having resealing capacity after being penetrated with a device for establishing fluid connection with the container.

A further object of the present invention is to eliminate the final sealing step of the front end of the port with a foil or a cap.

A still further object of the present invention is to provide a port with low risk of migration of potentially hazardous agents from the port to the stored fluids by avoiding direct contact between such polymeric materials that are at risk of migrating such agents and the stored fluid or by minimizing the utility of such materials.

A yet further object of the present invention is to provide a port which can be discarded with the remaining container for recycling without separate disengagement and collection.

These objects are attained by the present invention as disclosed in the description below and in the appended claims.

### DESCRIPTION OF THE INVENTION

A port system according to the present invention shall establish fluid communication with a container for storing medical fluids and a device for adding or withdrawing fluids from said container. Such a device typically is a syringe, a cannula, a spike connection to an infusion device or a device with similar function. The port system comprises at least one port having a sealed front end and a rear end open to the inside of the container and a base plate attachable to said container. Furthermore, the port has a penetrable membrane serving as a barrier to the stored fluids which can be penetrated by the device for establish fluid connection with the container. In its front end, the port comprises a cap that axially extends into an essentially sleeve formed part provided with said penetrable membrane which serves as partition to the stored fluids. The membrane is preferably placed at a given axial distance from the front end of the sleeve formed part which is shorter than its axial distance to the rear open end of said sleeve formed part. The cap seals the upper face of the penetrable membrane from contamination during handling and storage of port system as well as during its attachment to the container or the flexible film to be shaped as a container. Furthermore, the cap seals a protected space above said membrane which cannot be reached by microbial contamination. In order to open the port and make the penetrable membrane available for a device for establishing fluid communication, the port is provided with exposure means by which the user readily can uncover said membrane when it is desired to enter the container. referably, the exposure means partially or entirely removes the cap and it can consist of a zone, wholly or partially extending around the periphery of the port, having a predetermined weakness, so the user by a prescribed twisting motion readily can remove a part of the port along said zone. As will be described below in greater detail, such as zone preferably can consist of a groove extending along the outer periphery with a reduced material thickness which can be formed when molding the port system.

Alternative exposure means can of course be considered by the skilled person in the form of various frangible or rupturable constructions in order to remove a suitable part of the port.

It is an import aspect of the invention that the port system is molded in one part of a polyolefin material. The front sealing cap will extend directly into the sleeve formed part which directly extends into the base plate. By producing the port system in sealed mold at a temperature well above the sterilizing temperature, it is ascertained that the upper surface of the membrane and the space above enclosed by the front cap are unlikely to be reached by microbial contamination. This is of great advantage, since this part of the port system will not be reached by sterilizing steam during the autoclavation process to which it will be subjected when finally attached to the filled container.

According to a specific embodiment of the present invention, the penetrable and sealing membrane is connected to a stopper of an elastomeric material which can be regarded as an extension of said membrane that particularly adapts such a port to be entered by conventional syringe needles. The elastomer stopper entirely takes up a predetermined space between said membrane and the front end of the sleeve formed part of the port. As disclosed below, the stopper preferably is introduced in said space by injection molding with a heated, liquefied elastomer through an aperture in the sleeve formed part having size suitable for communication with a conventional injection nozzle, so a pierceable elastomeric stopper is formed through which repeated entries with a penetrating needle is possible without any subsequent leakage of fluid. It is of importance that the stopper fills the predetermined space completely and that the aperture is correctly closed so no channels for microbial transport inadvertently are formed after the production.

According to an alternative embodiment of the present invention, the port is provided with a membrane adapted to be penetrated with a spike of an infusion device. In this case the membrane is designed to facilitate the penetrative operation, for example by being provided with directing means for a correct penetration of the spike such as centrally intersecting grooves meeting in the central point of penetration. Furthermore, the membrane has a certain suitable thickness to enable a sealing action of the membrane even when penetrated by the spike throughout the administration of the infusion fluid which means that the port will be held upside down during gravity fed infusion. Preferably, the front end of the sleeve formed part is formed with a mouthpiece which is designed to fit conventional spikes. In order to readily distinguish the ports designated for spikes from ports designated for syringes when being parts of the same port system, the spike port preferably can have an essentially longer sleeve formed part and its periphery can be provided with an annular protruding flange.

In both the mentioned embodiments the cap serves as an effective protection means by sealing the penetrable front surface of the stopper or the membrane from contamination during the storage and handling of the port system.

It is conceivable and within the scope of protection to design the port systems according to the present invention with different combinations of the mentioned ports. Any combination of the mentioned ports adapted to syringes or spikes will be conceivable although a port system having one port of each category is exemplified below.

The present invention also pertains to a manufacturing method of a port system for medical flexible container of a polymeric material comprising at least one sealed port and a base plate, wherein the port system is made in one part in a closed mold by an injection molding step at a temperature above about 180° C., so as to form a port having an at least partly detachable front cap sealing the front surface of a membrane in a sleeve formed part axially extending from said cap to the base plate. referably a zone or a line of predetermined weakness in tie material extending around the port is formed already in said molding step in order to make the cap detachable with a simple twisting motion of the user.

The mold is basically of a conventional closed design for manufacturing hollow articles of a polymeric materials by injection molding and admits the introduction of different materials (i.e. two-color molding), as is also described in the Swedish Patent Application No. 9700597-9. Additionally, the mold is provided with means for removing the cores from the molded ports and with thermal sealing bars which serve to seal the front end of the port and to form its cap like front. The mentioned additional features of the mold are capable of operating within its closed system and are not described herein in further detail, since it is appreciated that its within the ability of the person skilled in this technique to design such a mold equipment. Principally, the inventive method of manufacturing the port system, as disclosed above, after it is initially formed in the mold includes the steps of:

a) letting the port system reach its setting temperature in the mold;

b) removing the cores from the molded port with a core pulling system;

c) while still in the closed mold, sealing the front end of the port so as to form the sealed front cap with the heated seal bars; and finally

d) releasing the port system from the mold.

It is an important aspect of the inventive method that the molded port system not allows to reach a temperature below about 60 to 80° C. from its setting temperature, preferably not below about 70° C.

In order to manufacture a port having an elastomer stopper connected to the upper surface of the protective membrane with the inventive method, liquefied elastomer can be introduced into the closed mold and into said predetermined space above said membrane, preferably by means of high pressure injection. In identity with what has been stated above, the port system has then reached its setting temperature and not is cooled below about 60 to 80° C., preferably not below about 70° C., before the liquefied elastomer is introduced into the port in the closed mold, preferably by high pressure injection, so as to form a resealable stopper of the elastomer, whereupon the port system is released from the mold. This is accomplished by injecting the liquefied elastomer through an aperture above the front surface of the membrane in the sleeve formed part of the port to completely fill a predetermined region of said sleeve formed part when forming the stopper. The injection of elastomer can into the port can be performed either before or after its front end is sealed with the heated bars (i.e. before or after step c), above). However, even if both alternatives are conceivable to the skilled person, it is preferred to inject the elastomer before sealing the cap in the mold.

Preferably, said elastomer is made with high pressure injection molding in accordance with the method disclosed in the mentioned Swedish latent Application No. 9700597-9, so a membrane is formed with a resealing capacity at least according to the requirements of the standard norm DIN 58 363. It is preferred to maintain a high sterilizing temperature of the port system during its removal from the mold until the heated introducing said elastomer entirely fills the predetermined space above the membrane in the sleeve formed part of the port. It is preferred that a sterilizing temperature (e.g. above 121° C.) is maintained during the manufacturing of the port system to accomplish a reduced risk of microbial contamination of any of its parts which in regular prescribed operations will be contacted by a device for establishing fluid connection with the container. This can be achieved by that the liquefied elastomer introduced in the high pressure molding has a temperature of above 180° C. and that the remaining port is molded by a material just reaching its setting temperature, but have been liquefied at a temperature above 180° C.

The so formed port system can now be attached to the container or to the flexible material to be shaped into container by means of welding. The container will thereafter be filled with a technique described in the Swedish Patent Application No. SE 9601348-7, finally sealed and sterilized, preferably with high pressure steam (autoclavation). The final sterilization will effectively sterilize the remaining surfaces of sleeve formed part of the port between the membrane and its rear opening which are in contact with the fluid of the container.

The inventive production process enables an effective way to seal off surfaces of the port system which normally are at risk for contamination and thereby providing port systems for flexible containers with higher safety while reducing the number production steps, in particular compared to processes where a sealing foil finally must be secured before a separate sterilization, usually with gamma radiation.

The port system preferably consists of medical grade polyolefins which may be compounded with a fraction of thermoplastic elastomer. The material of the port system must be possible to attach to the container for example by a simple welding process which means that there must be a compatibility to the material of the container. Preferably, the polyolefin is polypropylene or polyethylene based which means that essentially consists of polypropylene or polyethylene, optionally with a fraction copolymerized ethylene or propylene. Various medical grades of pure polypropylene or polyethylene are also conceivable materials

The elastomer material for production of the stopper preferably comprises a polyolelin compatible with the carrier and a thermoplastic elastomer. Suitable commercially available materials are Dynaflex® from GLS Corp., containing polypropylene and SEBS (styrene-ethylene-butadiene-styrene), Santoprene® containing polypropylene and EPDM-rubber, Evoprene® from Evode, and Craiwton®, as well as and various materials containing polyisobutylene (PIB). It is of importance that the stopper described in the present invention has a resealing capacity which fulfills at least the requirements of standard norm DIN 58 363 Part 15 that it should be reasealable after a penetration with a 0.6 mm needle without any escape of fluid.

The following detailed part of the description illustrates an embodiment of the present invention that should not be regarded as a limitation of the invention as disclosed in the claims.

### DETAILED DESCRIPTION OF THE INVENTION

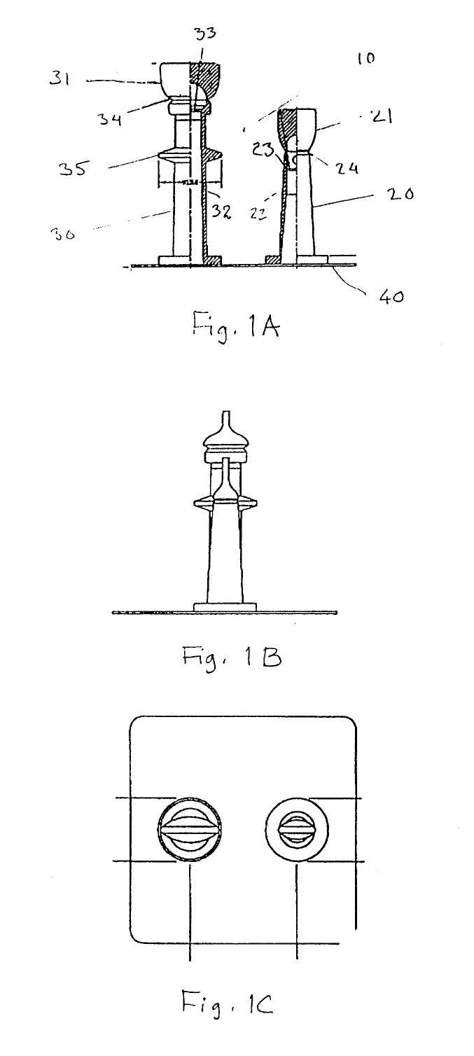

FIG. 1A shows a side view of a port system according to the present invention having a specific port for additives and a specific port for connection with a spike of an infusion set.

FIG. 1B shows a side view of a port system according to the present invention.

FIG. 1C shows a top of view of a port system according to the present invention.

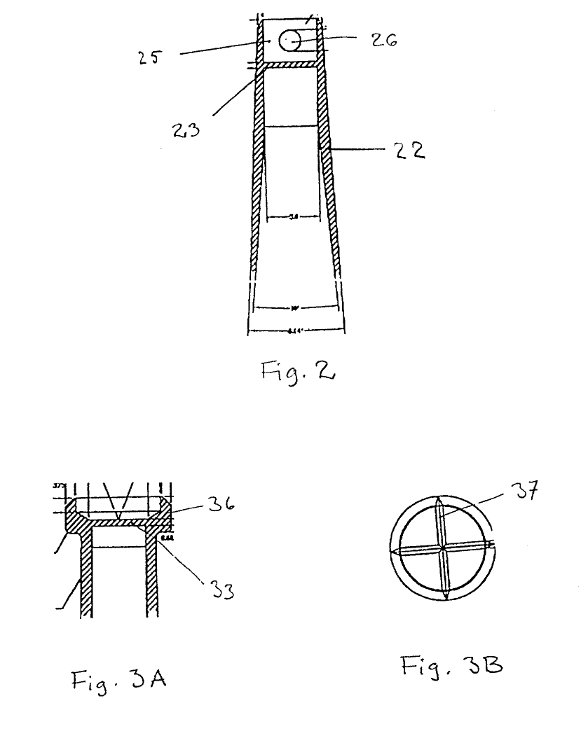

FIG. 2 shows a cross-sectional side view of an embodiment of a port according to the present invention.

FIG. 3A shows a cross-sectional side view of another embodiment of a port according to the present invention.

FIG. 3B shows a top view of the penetrable membrane of the port shown in FIG. 3A.

FIG. 1A shows an embodiment of a port system 10 according to the present invention comprising two different ports 20, 30 and a base plate 40 to be attached to a flexible container. The port 20 is an additive port through which additional agents are introduced to the fluids stored in the container. The port 30 is aimed to connected to a spike of an infusion set. The ports generally comprise a cap 21,31 in their front end which extends into a sleeve formed part 22,32 which is provided with a penetrable membrane 23,33 close to the front end of said sleeve formed part. Furthermore, the ports are provided with weakenings in the form grooves 24,34 of the material along which the user can remove the cap 21,31 with a simple, twisting motion to expose a surface for penetration with device to establish fluid connection with the container. In the embodiment shown of FIG. 1A, the two ports 20,30 is made easily distinguishable by having characteristically different sizes and the spike port 30 is also provided with a clearly characterizing flange 35 to simplify the identification of the ports for the user of a container.

As best demonstrated in FIG. 2, the sleeve formed part 22 of the port 20, for addition of a supplementary agent to the stored fluid, is provided with penetrable membrane 23 serving as a partition between the stored fluid and a penetrable stopper 25 made of an elastomer to avoid migration of potentially hazardous agents from the elastomer to fluid. The stopper 25 extends from the membrane to the front end of the sleeve formed and has a front surface that is protected from contamination by the cap. In order to avoid contamination in this region of the port, the stopper is introduced into the space between the membrane and the front end of the sleeve formed part by injection molding through an aperture 26 in said sleeve formed part, while maintaining the port system at high, sterilizing temperature before and during the molding of elastomer. For the same reason, it also important that said space is thoroughly and carefully filled with elastomer so the aperture is completely sealed and that no channels are formed in the stopper. During the storage of the container, the front surface of the elastomeric stopper is protected from contamination by the cap.

FIG. 3A shows a side view of the front part of the sleeve formed part 32 of the port 30 for connection with a spike device of a conventional infusion set. The membrane 33 is considerably thicker than in the addition port to enable a sealing capacity although the comparatively coarse penetrating means of the spike device penetrates the membrane. The front part of the sleeve formed is formed as a radially outwardly directed mouthpiece 36 to accomplish a convenient fitting with the spike device. As shown in FIG. 3B the membrane 33 can be provided with grooves to facilitate the direction of the spike to a suitable central point for penetration which also are capable of partial resealing after the spike has been displaced.

1. A port system for establishing fluid communication with a container for storing medical fluids and a penetrating device for adding or withdrawing fluids from said container comprising at least one port connected to base plate attachable to a container wall, said port is provided with a sealing membrane having
(i) a sealed front surface protected from contamination from its production moment until it is exposed to said penetrating device
(ii) a rear surface serving as a barrier to the fluids stored in the container;
to and wherein said port further is provided with a removable front cap which seals said front surface of the membrane.

2. A port system according to claim 1, wherein the port comprises, in one molded part made of a polyolefin material, the front sealing cap directly extending into a generally sleeve formed part further extending into the base plate.

3. A port system according to claim 2, wherein the sleeve formed part axially extends between an open front end an open rear end and is provided with the radially extended sealing membrane at a predetermined distance from said front end.

4. A port system according to claim 1 characterized in that the port is provided with a weakness in the material so the cap is at least partially removed to expose the penetrable front surface of the membrane.

5. A port system according to any of claims 1 to 4 characterized in that the membrane is connected to a stopper of an elastomeric material.

6. A port system according to claim 4 characterized in that the port is provided with an aperture for introducing the elastomeric material.

7. A port system according to claim 6 characterized in that the aperture is located between the front end of the sleeve formed part and the sealing membrane.

8. A port system according to claim 6 characterized in that the stopper is formed by high pressure injection molding.

9. A port system according to claim 5 characterized in that the stopper is resealable after multiple entries into the container with a penetrating device.

10. A port system according to claim 9 characterized in that the stopper has resilient capacity of at least the requirements of the standard norm DIN 58 363 (part 15).

11. A port system according to any of claims 1 to 10 consisting of polyolefin polymers to such an extent that it is possible to recycle in a single process.

12. A port system according to claim 11 characterized in that the polyolefin polymers comprises polypropylene or a copolymer thereof.

13. A port system having two ports connected to a base plate wherein a first port suitable for penetration of a syringe is provided with the elastomer stopper according to claims 5 to 12 and wherein a second port according to claims 1 to 4 has a longer sleeve formed part than said first port and is provided with an annular flange extending around the periphery of its sleeve formed part.

14. A port system according to claim 13 characterized in that the front surface of the membrane of its second port is provided with centrally intersecting grooves.

15. A port system according to claim 13 characterized in that the front end of the sleeve formed of the second part is formed with a radially outwardly directed mouthpiece.

16. A method of manufacturing a port system for medical flexible container of a polymeric material comprising at least one sealed port and a base plate, wherein the port system is made in one part in a closed mold by an injection molding step at a temperature exceeding about 180° C., so as to form the port having an at least partly detachable front cap sealing the front surface of a membrane in a sleeve formed part axially extending from said cap to the base plate.

17. A method according to claim 16 characterized by
(i) letting the port system reach its setting temperature in the mold;
(ii) while still in the closed mold, sealing the front end of said port so as to form the front cap;
(iii) releasing the port system from the mold.

18. A method according to claim 17 characterized by while still in the closed mold, injecting the liquefied elastomer through an aperture above the front surface of the membrane in the sleeve formed part and thereby completely filling a predetermined region of said sleeve formed part when forming the stopper.

19. A method according to claim 16 characterized by maintaining the molded port system at a temperature above at least 121° C.

20. A method according to claim 16 characterized by molding a zone of predetermined weakness in the material extending around the port so the cap is detachable.
